(19) United States
(12) Patent Application Publication
(10) Pub. No.: US 2006/0078576 A1
(43) Pub. Date: Apr. 13, 2006

(54) Method for producing hydronycarboxylic acid esters

(76) Inventors: Ansgar Behler, Bottrop (DE); Almud Folge, Lechlingen (DE)

Correspondence Address: COGNIS CORPORATION;PATENT DEPARTMENT, 300 BROOKSIDE AVENUE, AMBLER, PA, 19002, US

(21) Appl. No.: 10/539,730

(22) PCT Filed: Dec. 10, 2003
(86) PCT No.: PCT/EP03/13978

§ 371 Date: Jun. 20, 2005

(30) Foreign Application Priority Data
Dec. 19, 2002 (DE) 10260131.3

(51) Int. Cl. A61K 8/37; C07C 67/08

(52) U.S. Cl. 424/401; 560/185; 554/174

(57) ABSTRACT

The invention relates to mixtures of mono-, di-, and tri-esters of hydroxycarboxylic acids and to a process for producing them. The hydroxycarboxylic acid is preferably citric acid. In the disclosed process, hydroxycarboxylic acids or salts thereof are reacted with a mixture of alcohols corresponding to general formulae (I) R¹—OH and (II) R²—(C₂H₄)ₙ—OH, where R¹ and R² independently of one another represent a saturated or unsaturated, branched or unbranched C₆₋₂₂ alkyl group and n is a number of 1 to 20, at temperatures of 120 to 180° C., wherein the compounds of formulae (I) and (II) are used in a ratio by weight of 10:1 to 1:10, and preferably in a ratio of 10:1 to 1:1.

This invention relates to a process for the production of selected hydroxycarboxylic acid esters and mixtures of hydroxycarboxylic acid esters and to their use in cosmetic compositions.

Hydroxycarboxylic acids are well-known compounds. Monoalkylhydroxycarboxylic acid esters in particular have long been used in cosmetic preparations. Thus, EP 282 289 A1, for example, describes a cosmetic composition containing monoalkylcitric acid salts. Besides the pure alkyl compounds, alkyl oxide compounds of citric acid are also disclosed in that document.

WO 94/10970 describes a solubilizer containing monoalkyl citrates with C₇₋₁₀ alkyl groups as an ingredient of perfumes, cosmetic compositions, such as body cleansing and care preparations, and textiles. DE 199 45 478 A1 describes cosmetic and/or pharmaceutical preparations which, besides alkyl and/or alkenyl oligoglycosides, contain hydroxycarboxylic acid partial esters or salts thereof.

Unfortunately, the known products are attended by various disadvantages. Thus, the known alkyl citrates often cannot be made up into clear formulations in conjunction with anionic surfactants and, even when they are combined with certain nonionic surfactants, such as alkyl polyglycosides, formulation problems can still arise. In addition, the pure alkyl citrates are present as high-melting pastes at room temperature. Accordingly, the problem addressed by the present invention was to overcome the disadvantages of known alkyl citrates and hydroxycarboxylic acid esters. It has been found that alkyl citrates without any of the disadvantages mentioned above can be obtained through the choice of the alcohol component.

The present invention also relates to a process for the production of hydroxycarboxylic acid esters, in which hydroxycarboxylic acids or hydroxycarboxylic acid salts are reacted with a mixture of alcohols corresponding to general formula (I) R¹—OH and (II) R²—(C₂H₄)ₙ—OH, where R¹ and R² independently of one another represent a saturated or unsaturated, branched or unbranched C₆₋₂₂ alkyl group and n is a number of 1 to 20, at temperatures of 120 to 180° C., characterized in that the compounds of formulae (I) and (II) are used in a ratio by weight of 10:1 to 1:10. The present invention also relates to the compounds obtained which are mixtures of various isomeric esters.

In principle, the process according to the invention may be carried out using any hydroxycarboxylic acids, particularly preferred hydroxycarboxylic acids being selected from the group consisting of lactic acid, tartaric acid, malic acid and citric acid and self-condensation products thereof. Citric acid is particularly preferred for the purposes of the invention.

The partial esters of hydroxy carboxylic acids in the context of the invention are surfactants which, preferably, still contain a free carboxyl group. Accordingly, the partial esters may also be acidic esters or neutralization products thereof. In that case, the partial esters are present in the form of alkali metal, alkaline earth metal, ammonium, alkylammonium, alkanolammonium and/or glucammonium salts.

The esters themselves are preferably derived from fatty alcohols of formula (I) which are used in admixture with ethoxylated fatty alcohols corresponding to general formula (II). According to the invention, the ratio by weight between the alcohols of formula (I) and the ethoxylated alcohols of formula (II) must be in the range from 10:1 to 1:10. In a particularly preferred embodiment, the alcohols of formula (I) and (II) are used in a ratio by weight of 10:1 to 1:1, more particularly 9:1 to 1:1, preferably 4:1 to 1:1 and most particularly 1:1.

The esters according to the invention are polyesters because several carboxyl functions can be esterified. Typically, the esters are in the form of mixtures from their production, of which about 25 to 30% may be formed by monoesters, 30 to 40% by diesters and 5 to 15% by triesters. The balance to 100% is formed by free hydroxycarboxylic acid.

Accordingly, the present invention also relates to mixtures of isomeric compounds corresponding to general formula (III): 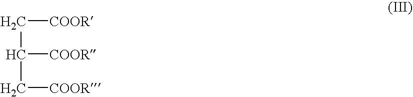
in which R′, R″, R′″ represent a hydrogen atom and/or a C₆₋₂₂ alkyl group and/or an ethoxylated C₆₋₂₂ alkyl group, the ethoxylated alkyl groups containing 2 to 20 parts ethylene oxide per alkyl group, with the provisos that at least one of the substituents R′, R″ and R′″ represents such an ethoxylated alkyl group and the total number of ethylene oxide units per ester molecule is limited to 20. The mixtures contain mono-, di- and triesters alongside one another, mono- and diesters preferably being present in a ratio of 1:1 to 1:2. The percentage content of free citric acid may be up to 20%, based on the mixtures. However, the mixtures preferably contain less free citric acid, preferably less than 10%.

The production process as such corresponds to the prior art. It can be essential to carry out the reaction in a nitrogen atmosphere. In addition, it can be of advantage to carry out the reaction at temperatures of 150 to 170° C. and preferably at 160° C. The monoalkylesters of the hydroxycarboxylic acids according to the invention are obtained as the end product and may be removed from the mixture, for example by distillation. The esters may be present in free form or as salts. A small percentage of the hydroxycarboxylic acid, preferably at most 20% by weight and more particularly at most 10% by weight, remains unesterified in the process. Reaction products containing at most 8% and more particularly at most 5% unesterified citric acid are particularly preferred.

The acid value (DIN 51963) of the products obtained in accordance with the invention is preferably in the range from 200 to 300 while their OH value is preferably in the range from 180 to 250, their ester value is preferably in the range from 100 to 160 and their saponification value is preferably in the range from 380 to 500 (all measurements to DIN).

The hydroxycarboxylic acid esters produced in accordance with the invention and preferably the esters of citric acid may advantageously be formulated with anionic and/or nonionic surfactants to form aqueous solutions.

Particularly preferred nonionic surfactants are inter alia fatty alcohols, alcohol ethoxylates and alkyl polyglycosides. Particularly suitable anionic surfactants are alkyl ether sulfates although the choice of the anionic surfactants is not limited to alkyl ether sulfates.

Fatty Alcohols

The fatty alcohols which are also used in the synthesis of the hydroxycarboxylic acids according to the invention correspond to formula (I):
R¹OH (I)
where R¹ is an aliphatic, linear or branched hydrocarbon radical containing 6 to 22 carbon atoms and 0 and/or 1, 2 or 3 double bonds. Typical examples are caproic alcohol, caprylic alcohol, 2-ethylhexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol and the technical mixtures thereof obtained, for example, in the high-pressure hydrogenation of technical methyl esters based on fats and oils or aldehydes from Roelen's oxo synthesis and as monomer fraction in the dimerization of unsaturated fatty alcohols. Preferred fatty alcohols are technical C₁₂₋₁₈ fatty alcohols such as, for example, coconut oil, palm oi, palm kernel oil or tallow fatty alcohol.
Alcohol Ethoxylates

Alcohol ethoxylates are known as fatty alcohol or oxoalcohol ethoxylates from their production and preferably correspond to formula (II):
R²O(CH₂CH₂O)ₙH (II)
in which R² is a linear or branched alkyl and/or alkenyl group containing 6 to 22 carbon atoms and n is an integer of 1 to 50. Compounds of formula (II) with a degree of ethoxylation of 1 to 20 are used for the synthesis in the process according to the invention for the production of hydroxycarboxylic acid esters. Typical examples are the adducts of on average 1 to 20, preferably 1 to 10 and more particularly 1 to 5 mol ethylene oxide with caproic alcohol, caprylic alcohol, 2-ethylhexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol and brassidyl alcohol and the technical mixtures thereof obtained, for example, in the high-pressure hydrogenation of technical methyl esters based on fats and oils or aldehydes from Roelen's oxo synthesis and as monomer fraction in the dimerization of unsaturated fatty alcohols. Adducts of 1 to 10 mol ethylene oxide with technical C₁₂₋₁₈ fatty alcohols such as, for example, coconut oil, palm oil, palm kernel oil or tallow fatty alcohol are preferred.
Alkyl and/or Alkenyl Oligoglycosides

Alkyl and alkenyl oligoglycosides are known nonionic surfactants corresponding to formula (IV):
R³O—[G]ₚ (IV)
where R³ is an alkyl and/or alkenyl group containing 4 to 22 carbon atoms, G is a sugar unit containing 5 or 6 carbon atoms and p is a number of 1 to 10. They may be obtained by the relevant methods of preparative organic chemistry. The alkyl and/or alkenyl oligoglycosides may be derived from aldoses or ketoses containing 5 or 6 carbon atoms, preferably glucose. Accordingly, the preferred alkyl and/or alkenyl oligoglycosides are alkyl and/or alkenyl oligoglucosides. The index p in general formula (IV) indicates the degree of oligomerization (DP), i.e. the distribution of mono- and oligoglycosides, and is a number of 1 to 10. Whereas p in a given compound must always be an integer and, above all, may assume a value of 1 to 6, the value p for a certain alkyl oligoglycoside is an analytically determined calculated quantity which is generally a broken number. Alkyl and/or alkenyl oligoglycosides having an average degree of oligomerization p of 1.1 to 3.0 are preferably used. Alkyl and/or alkenyl oligoglycosides having a degree of oligomerization of less than 1.7 and, more particularly, between 1.2 and 1.4 are preferred from the applicational point of view. The alkyl or alkenyl radical R³ may be derived from primary alcohols containing 4 to 11 and preferably 8 to 10 carbon atoms. Typical examples are butanol, caproic alcohol, caprylic alcohol, capric alcohol and undecyl alcohol and the technical mixtures thereof obtained, for example, in the hydrogenation of technical fatty acid methyl esters or in the hydrogenation of aldehydes from Roelen's oxosynthesis. Alkyl oligoglucosides having a chain length of C₈ to C₁₀ (DP=1 to 3), which are obtained as first runnings in the separation of technical C₈₋₁₈ coconut oil fatty alcohol by distillation and which may contain less than 6% by weight of C₁₂ alcohol as an impurity, and also alkyl oligoglucosides based on technical C_{9/11} oxoalcohols (DP=1 to 3) are preferred. In addition, the alkyl or alkenyl radical R³ may also be derived from primary alcohols containing 12 to 22 and preferably 12 to 14 carbon atoms. Typical examples are lauryl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol and technical mixtures thereof which may be obtained as described above. Alkyl oligoglucosides based on hydrogenated C_{12/14} coconut oil fatty alcohol having a DP of 1 to 3 are preferred.
Alkyl Ether Sulfates

Alkyl ether sulfates (“ether sulfates”) are known anionic surfactants which, on an industrial scale, are produced by SO₃ or chlorosulfonic acid (CSA) sulfation of fatty alcohol or oxoalcohol polyglycol ethers and subsequent neutralization. Ether sulfates suitable for use in accordance with the invention correspond to formula (V):
R⁴O—(CH₂CH₂O)ₘSO₃X (V)
in which R⁴ is a linear or branched alkyl and/or alkenyl group containing 6 to 22 carbon atoms, m is a number of 1 to 10 and X is an alkali metal and/or alkaline earth metal, ammonium, alkylammonium, alkanolammonium or glucammonium. Typical examples are the sulfates of addition products of on average 1 to 10 and more particularly 2 to 5 mol ethylene oxide onto caproic alcohol, caprylic alcohol, 2-ethylhexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol and brassidyl alcohol and technical mixtures thereof in the form of their sodium and/or magnesium salts. The ether sulfates may have both a conventional homolog distribution and a narrow homolog distribution. It is particularly preferred to use ether sulfates based on adducts of on average 2 to 3 mol ethylene oxide with technical C_{12/14} or C_{12/18} coconut fatty alcohol fractions in the form of their sodium and/or magnesium salts.

The surfactant mixtures according to the invention may be used for the production of cosmetic preparations such as, for example, hair shampoos, hair lotions, foam baths, shower baths, creams, gels, lotions, alcoholic and aqueous/alcoholic solutions, emulsions, wax/fat compounds, stick preparations, powders or ointments. The hydroxycarboxylic acid esters according to the invention may also be used in combination with other auxiliaries and additives typically used for cosmetic preparations such as, for example, mild surfactants, oil components, emulsifiers, superfatting agents, pearlizing waxes, consistency factors, thickeners, polymers, silicone compounds, fats, waxes, lecithins, phospholipids, stabilizers, biogenic agents, deodorants, antiperspirants, antidandruff agents, film formers, swelling agents, UV protection factors and the like. Another embodiment of the present invention is the use of hydroxycarboxylic acid esters produced by the process claimed in claim 1 as auxiliaries for the production of cosmetic preparations, preferably those containing either alkyl ether sulfates or alkyl oligoglycosides or fatty alcohol ethoxylates or mixtures of these surfactants.

It can be of advantage to use mixtures of APG compounds corresponding to formula (IV) with the hydroxycarboxylic acid esters according to the invention for the production of cosmetic preparations in which the ratio by weight of the APGs to the hydroxycarboxylic acid esters or mixtures thereof is in the range from 3:1 to 1:3. Aqueous formulations are particularly preferred, particularly if they are mildly acidic and preferably have a pH of5to 6.5.

### EXAMPLES

1. Preparation of a Monoalkylcitric Acid

299.6 g (1.6 mol) of a C₁₂ fatty alcohol were introduced into a stirred vessel with a water separator together with 127.2 g (0.4 mol) of a C₁₂₋₁₄ fatty alcohol, which had been reacted with 3 mol ethylene oxide per mol fatty alcohol, and 384.2 g (2.0 mol) of water-free citric acid and heated under nitrogen to 160° C. On completion of the reaction, the reaction mixture was cooled to 100° C. and the end product was obtained by distillation. A yellow, viscous bright ester was obtained (yield 775.0 g).

The saponification number was 432, the acid value measured 284, the OH value was 210, the ester value was 148. The percentage content of free citric acid was 3.7% by weight.

2. Performance Tests

A mixture of an ether sulfate (Texapon N, a product of Cognis Deutschland GmbH & Co. KG) both with pure lauryl citrates (C1) and with lauryl-/C₁₂₋₁₄ fatty alcohol+3EO citrate in a ratio by weight of 3:1 or 9:1 (E1 and E4) (based on active substance) in water was prepared, the pH of the solution being adjusted to 6.

Results:

| | | |
|---|---|---|
| C1 | Lauryl citrate | Cloudy, separating liquid |
| 2 | Lauryl-C₁₂₋₁₄ fatty alcohol + | Clear, bright liquid |
| | 3EO citrate (9:1) | |
| C3 | Lauryl citrate + C₁₂₋₁₄ fatty | Cloudy, separating liquid |
| | alcohol + 3EO citrate (9:1) | |
| 4 | Lauryl-C₁₂₋₁₄-fatty alcohol + | Clear, bright liquid |
| | 3EO citrate (3:1) | |

It is clear that only the products according to the invention lead to useful products; even the subsequent mixing of alkyl and alkyloxy citrates (C3) does not produce the required result.

In addition, a commercially available alkyl (oligo)glycoside (APG), Plantacare® 1200 of Cognis Deutschland GmbH & Co. KG, was made up into an aqueous formulation with various alkyl citrates (pH 6). The ratio by weight of APG to alkyl citrate was 3:1. The total active substance content of the solutions was 30%.

Results:

| | |
|---|---|
| Lauryl citrate | Inhomogeneous cloudy dispersion |
| Lauryl-C₁₂₋₁₄-fatty alcohol + | Homogeneous, slightly opaque solution |
| 3EO citrate (9:1) | |
| Lauryl-C₁₂₋₁₄-fatty alcohol + | Homogeneous, slightly opaque solution |
| 3EO citrate (3:1) | |

1-10. (canceled)

11. A process for the production of a mixture of mono-, di- and triesters of hydroxycarboxylic acids, said process comprising reacting hydroxycarboxylic acids or salts thereof with a mixture of alcohols corresponding to the formulas (I) R¹—OH and (II) R²—(C₂H₄)ₙ—OH, where R¹ and R² independently represent a saturated or unsaturated, branched or unbranched C₆₋₂₂ alkyl group and n is a number of 1 to 20, wherein the compounds of formulas (I) and (II) are used in a ratio by weight of 10:1 to 1:1 and the hydroxycarboxylic acid is used in such quantities that the molar ratio of the free carboxyl groups of the hydroxycarboxylic acid to the free OH groups of the alcohols is at most 3.5:1.

12. A process according to claim 11, wherein the alcohols of formulas (I) and (II) are used in a ratio by weight of 4:1 to 1:1.

13. A process according to claim 11, wherein the alcohols of formulas (I) and (II) are used in a ratio by weight of 1:1.

14. A process according to claim 11, wherein the hydroxycarboxylic acid is used in such quantities that the molar ratio of the free carboxyl groups of the hydroxycarboxylic acid to the free OH groups of the alcohols is at most 3:1.

15. A process according to claim 11, wherein the reaction is carried out at temperatures of 150 to 170° C.

16. A process according to claim 11, wherein the hydroxycarboxylic acid is selected from the group consisting of lactic acid, tartaric acid, malic acid, citric acid and self-condensation products thereof.

17. A process according to claim 16, wherein the hydroxycarboxylic acid is citric acid.

18. A process according to claim 11, wherein the resulting mixture of hydroxycarboxylic acid esters contains 25 to 30% of monoesters, 30 to 40% of diesters, 5 to 15% by triesters, and up to 8% of free hydroxycarboxylic acid.

19. A mixture of mono-, di- and triesters of hydroxycarboxylic acids, and optionally free, hydroxycarboxylic acid, the mixture comprising esters corresponding to the formula (III): 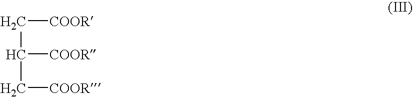
in which R′, R″, R′″ represent a hydrogen atom, a C₆₋₂₂ alkyl group, or an ethoxylated C₆₋₂₂ alkyl group, the ethoxylated alkyl groups containing 2 to 20 parts ethylene oxide per alkyl group, with the provisos that at least one of the substituents R′, R″ and R′″ represents such an ethoxylated alkyl group, and wherein the ratio of mono- to diesters in the mixture is 1:1 to 1:2.

20. A mixture according to claim 19, wherein the ethoxylated alkyl groups contain 2 to 10 parts ethylene oxide per alkyl group.

21. A mixture according to claim 19, wherein the hydroxycarboxylic acid is selected from the group consisting of lactic acid, tartaric acid, malic acid, citric acid and self-condensation products thereof.

22. A mixture according to claim 19, wherein the hydroxycarboxylic acid is citric acid.

23. A mixture according to claim 19, wherein the ethoxylated alkyl groups contain 2 to 10 parts ethylene oxide per alkyl group and the hydroxycarboxylic acid is citric acid.

24. A mixture according to claim 19, consisting of 25 to 30% of monoesters, 30 to 40% of diesters, 5 to 15% by triesters and up to 8% of free hydroxycarboxylic acid.

25. A cosmetic composition comprising
(A) one or more cosmetically active ingredients and
(B) a mixture of mono-, di- and triesters of hydroxycarboxylic acids, and optionally free, hydroxycarboxylic acid, the mixtures containing esters corresponding to the formula (III): 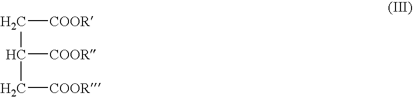
in which R′, R″, R′″ represent a hydrogen atom, a C₆₋₂₂ alkyl group, or an ethoxylated C₆₋₂₂ alkyl group, the ethoxylated alkyl groups containing 2 to 20 parts ethylene oxide per alkyl group, with the provisos that at least one of the substituents R′, R″ and R′″ represents such an ethoxylated alkyl group, and wherein the ratio of mono- to diesters in the mixture is 1:1 to 1:2.

26. A cosmetic composition according to claim 25, wherein the ethoxylated alkyl groups of component (B) contain 2 to 10 parts ethylene oxide per alkyl group.

27. A cosmetic composition according to claim 25, wherein the hydroxycarboxylic acid of component (B) is citric acid.

28. A cosmetic composition according to claim 25, wherein, in component (B), the ethoxylated alkyl groups containing 2 to 10 parts ethylene oxide per alkyl group and the hydroxycarboxylic acid is citric acid.

29. A cosmetic composition according to claim 25, the mixture of component (B) consists of 25 to 30% of monoesters, 30 to 40% of diesters, 5 to 15% by triesters and up to 8% of free hydroxycarboxylic acid.

30. A cosmetic composition according to claim 25, further comprising alkyl ether sulfates, alkyl oligoglycosides or fatty alcohol ethoxylates or mixtures thereof.
